(19) United States
(12) Patent Application Publication
(10) Pub. No.: US 2011/0038996 A1
(43) Pub. Date: Feb. 17, 2011

(54) 1-MONOPROPIONINE COMPOUND AND ITS ISOMER 3-MONOPROPIONINE AS PRESERVING AGENTS FOR ANIMAL FEED, GRAINS AND ANIMAL-ORIGIN MEALS

(76) Inventor: Paulo Roberto De Lima Portilho, Santa Barbara do Oeste (BR)

Correspondence Address: Mark Levy;HINMAN, HOWARD & KATTELL, LLP, 80 Exchange Street, P.O. Box 5250, BINGHAMTON, NY, 13901, US

(21) Appl. No.: 12/611,224

(22) Filed: Nov. 3, 2009

(30) Foreign Application Priority Data
Aug. 17, 2009 (BR) 015090002468

(51) Int. Cl. A23K 3/00; C07C 53/00

(52) U.S. Cl. 426/332; 562/606; 426/335

(57) ABSTRACT

“1-MONOPROPIONINE COMPOUND AND ITS ISOMER 3-MONOPROPIONINE AS PRESERVING AGENTS FOR ANIMAL FEED, GRAINS AND ANIMAL-ORIGIN MEALS” refers to the technical field of animal feed products, more specifically a 1-monopropionine compound and its isomer 3-monopropionine as preserving agents for animal feed, grains and animal-origin meals, with the object to reduce the contamination by deleterious fungi, yeasts and bacteriae (especially Salmonella spp and Coli spp) in animal feed, grains and animal-origin meals, and consequently enhance the growth of the animal by means of practically contaminant-free feeding.

### PRESENTATION

The present invention refers to the technical field of animal feed products, more specifically a 1-monopropionine compound and its isomer 3-monopropionine as preserving agents for animal feed, grains and animal-origin meals, with the object to reduce the contamination by deleterious fungi, yeasts and bacteria (especially Salmonella spp and Coli spp) in animal feed, grains and animal-origin meals, and consequently enhance the growth of the animal by means of practically contaminant-free food.

### BACKGROUND

The use of organic acids as preserving agents for animal feed, grains and animal-origin meals has been known for long. However, there are great inconveniences caused by the use of said technology, among which: the corrosiveness of said chemical agents, their volatility and, in some cases, mucosa irritation which may be caused in operators of manufacturing processes.

### STATE OF THE ART

The vast majority of natural product applications to reduce microbiological contamination in animal feed, grains and animal-origin meals is made with pure organic acids or their corresponding salts of calcium, sodium, potassium or ammonium (e. g. ammonium propionate, calcium propionate, potassium formate and others).

### Problems and Difficulties in the State of the Art

The action of pure acids is intense against bacteriae, fungi and yeasts, but is followed by great corrosiveness and risk, both for the equipments used and for their operators. This problem is partially solved when salts of acids are used, but their activity is 100 to 1000 times lower than their corresponding acids (e.g. propionic acid in comparison with calcium propionate).

### Featured Enhancements

Bearing in mind the demand to solve the presented problems, pure organic acids or their corresponding salts of calcium have been substituted with a non-corrosive, non-volatile and absolutely safe compound for processing equipment of animal feed, grains and animal-origin meals. They are also more efficient to control bacterial and fungal growth than equivalent doses of equivalent commercial products based on propionic acid or its ammonia salt.

The enhancement offered by the compound over the control of bacterial and fungal growth may be proven by means of experimental data, wherein samples of animal feed were produced under different concentrations of antimicrobial preserving agents and submitted to fungal challenge (inoculation with spores) and bacterial challenge (inoculation with bacteriae) with later analysis to verify the reduction or not of fungal and bacterial load.

### DISCLOSURE OF THE INVENTION

The invention is the application of a 1-monopropionine compound and its isomer 3-monopropionine as preserving agents for animal feed, grains and animal-origin meals.

Monopropionine is obtained by the reaction process of sterification of propionic acid on positions 1 or 3 of carbon in glycerol, undertaken under high temperature (preferably 280° C.) and vacuum. This is a translucent viscous liquid with low melting point (8° C.), preferably containing between 25% and 52% of propionic acid in its final constitution. This product may be used both pure and diluted in other liquids, or even sprayed on an absorbing substance (usually highly active clay or silica) to obtain a powdered product.

Objects, advantages and further important characteristics of the invention at issue may be more easily understood when read jointly with the formulation and obtaining process.

### DETAILED DESCRIPTION OF THE INVENTION

The present invention for “Monopropionine Compound as a Preserving Agents for Animal Feeds, Grains and Animal-Origin meals” is used as a preserving agent for food products for animals, being comprised by a monopropionine compound as applied over animal feed, grains and animal-origin meals, having the following preferential mixtures:
0.05 to 1% of the monopropionine compound in the quantity of the animal feed mixture;
more preferably, 0.1 to 0.3% of the monopropionine compound in the quantity of the animal feed mixture;
0.05 to 1% of the monopropionine compound in the quantity of the mixture of grains;
more preferably, 0.1 to 0.3% of the monopropionine compound in the quantity of the mixture of grains;
0.05 to 1% of the monopropionine compound in the quantity of the mixture of animal-origin meals;
more preferably, 0.1 to 0.3% of the monopropionine compound in the quantity of the mixture of animal-origin meals;

However, measurements, quantities and values may vary according to the requirements of use and type of animal feed, grains and animal-origin meals, as long as the same characteristics are maintained.

The monopropionine compound may be:
distilled monopropionine with 30% to 55% propionic acid in glycerol;
monopropionine with 30% to 42% propionic acid with 6% to 12% free acid in glycerol;
monopropionine with 30% to 42% propionic acid and 6% to 12% sodium propionate in glycerol;
monopropionine with 30% to 42% propionic acid and 6% to 12% potassium propionate in glycerol; and
monopropionine with 25% to 42% propionic acid and 6% to 12% ammonium propionate in glycerol.

Monopropionine might be mixed with the final product in pure form or even diluted in glycerol or water (liquids), combined with ammonia, sodium or potassium salts of propionic acid (liquid) and adsorbed with silica (powder).

The mixture of monopropionine compound with animal feeds, grains or animal-origin animal feeds is made in traditional mixers, following previously determined dosages.

The present invention reduces the contamination by deleterious fungi, yeasts and bacteriae in animal feeds, grains and animal-origin meals, consequently enhancing animal growth by means of practically contaminant-free feed.

All these properties allow to classify the 1-monopropionine compound and its isomer 3-monopropionine as preserving agents for animal feed, grains and animal-origin meals, a fully efficient and safe product for use to produce animal feeds, to protect grains and animal-origin meals and presenting excellent quality in final products. Therefore, the monopropionine compound will be well received by animal food product industries, which will be able to improve the quality of final products.

1. A 1-monopropionine compound and its isomer 3-monopropionine for use as preserving agents for animal feed, grains and animal-origin meals.

2. Compound of claim 1, characterized by the monopropionine being distilled with 30% to 55% propionic acid in glycerol.

3. Compound of claim 1, characterized by the monopropionine having 30% to 42% propionic acid with 6% to 12% free acid in glycerol.

4. Compound of claim 1, characterized by the monopropionine having 30% to 42% propionic acid with 6% to 12% sodium propionate in glycerol.

5. Compound of claim 1, characterized by the monopropionine having 30% to 42% propionic acid with 6% to 12% potassium propionate in glycerol.

6. Compound of claim 1, characterized by the monopropionine having 25% to 42% propionic acid with 6% to 12% ammonium propionate in glycerol.

7. Compound of claim 1, characterized by the preferential mixture of 0.05 to 1% of monopropionine compound in the quantity of animal feed.

8. Compound of claim 7, characterized by the more preferential mixture of 0.1 to 0.3% of monopropionine compound in the quantity of animal feed.

9. Compound of claim 1, characterized by the preferential mixture of 0.05 to 1% of monopropionine compound in the quantity of grains.

10. Compound of claim 9, characterized by the more preferential mixture of 0.1 to 0.3% of monopropionine compound in the quantity of grains.

11. Compound of claim 1, characterized by the preferential mixture of 0.05 to 1% of monopropionine compound in the quantity of animal-origin meals.

12. Compound of claim 11, characterized by the more preferential mixture of 0.1 to 0.3% of monopropionine compound in the quantity of animal-origin meals.

13. Compound of claim 7, characterized by the measurements, quantities and values varying according to the type of animal feed, grains and animal-origin meals.

14. Compound of claim 1, characterized by the monopropionine being mixed with the final product in pure form or even diluted in glycerol or water (liquids), combined with ammonia, sodium or potassium salts of propionic acid (liquid) and adsorbed with silica (powder).

15. Compound of claim 12, characterized by the measurements, quantities and values varying according to the type of animal feed, grains and animal-origin meals.
